(19) United States
(12) Patent Application Publication
(10) Pub. No.: US 2025/0235677 A1
(43) Pub. Date: Jul. 24, 2025

(54) NASAL DRUG DELIVERY DEVICE WITH ADJUSTABLE POSITION

(71) Applicant: Nasal-Phyto (SZ) Pharmaceutical Technology Co., Ltd., Shenzhen, Guangdong (CN)

(72) Inventors: Jingyi Liu, Shanghai (CN); Jian Zhou, Shenzhen, Guangdong (CN); Wei Liu, Shenzhen, Guangdong (CN)

(21) Appl. No.: 18/549,590

(22) PCT Filed: Jun. 16, 2023
(86) PCT No.: PCT/CN2023/100865

§ 371 Date: Sep. 8, 2023

(30) Foreign Application Priority Data
Oct. 11, 2022 (CN) 202222676570.5

(51) Int. Cl. A61M 31/00

(52) U.S. Cl. CPC A61M 31/00; A61M 2202/0007; A61M 2205/0216; A61M 2205/10; A61M 2210/0618

(57) ABSTRACT

The disclosure provides a nasal drug delivery device with adjustable position comprising: an intranasal locking connector, a nasal dropper, a syringe pump and a syringe. One end of the nasal dropper is movably set through the intranasal locking connector, and the nasal dropper is fixed by the intranasal locking connector, and the other end of the nasal dropper is connected to the output end of the syringe. The syringe is connected to the syringe pump. The split structure of the nasal dropper and the intranasal locking connector are adopted, the nasal dropper is movably set in the intranasal locking connector and fixed by the intranasal locking connector, when the intranasal locking connector is fixed under the user's nasal cavity, by adjusting the length of the nasal dropper in the nasal cavity, the drip position can be adjusted so as to be suitable for different patients.

### TECHNICAL FIELD

The disclosure relates to the field of medical equipment, in particular, relates to a nasal drug delivery device with adjustable position.

### BACKGROUND

The traditional medication for nervous system diseases is mainly oral and injection. In some cases, the effect of oral drugs is not good, especially for users who have difficulty in swallowing, such as the elderly and young children. Second, the treatment of brain diseases requires a high drug concentration in brain tissue. However, due to the first-pass effect of the liver and the existence of the blood-brain barrier, it is difficult to reach a high concentration in the brain tissue after oral administration. The third is that the disease is serious and progresses rapidly, requiring emergency treatment, while the traditional oral medicine takes a long time to reach the brain tissue and has a slow onset of effect. Fourth, the user has poor compliance with oral therapy, in these cases, the current solution is usually to take injection. Injection is an invasive administration. There are risk factors in the pharmaceutical preparations, such as insoluble particles, heat sources, microorganisms, etc., and they will cause higher clinical risks to users. In addition, the long-term injection of drugs may lead to poor compliance of users, which may cause psychological trauma to users.

Nasal administration has a natural clinical advantage under the above circumstances. First of all, this is a non-invasive way of drug delivery, and the clinical risk introduced to the user is low. The drug is absorbed into the blood or brain through the nasal mucosa. There is a natural immune barrier in the nasal environment and nasal mucosa, which can prevent pathogenic microorganisms or insoluble particles entering the body. Secondly, for users who have difficulty swallowing or are not suitable for injection administration, the use of nasal administration can make up for the shortcomings of the current traditional clinical administration methods, bringing more convenience and comfort to users.

Moreover, the nasal cavity administration in this disclosure can improve the brain-targeting of the drug, make the drug quickly reach the action site, reduce the concentration of the drug in the peripheral circulation, and reduce peripheral side effects. This new method delivers the drug directly to the brain through the nasal olfactory area in the form of a spray, providing users with a more convenient way to use, as well as more reliable and effective.

There are some problems when nasal drip administration is applied to inpatients. The current common nasal drip device has a fixed delivery point, and when the delivery point in the nasal cavity needs to be adjusted, the position of the nasal drip device can only be adjusted as a whole, but this does not facilitate the fixation of the nasal drip device.

### BRIEF SUMMARY

In response to the defects in the prior art, it is an object of the present disclosure to provide a nasal drug delivery device with adjustable position.

The disclosure provides a nasal drug delivery device with adjustable position comprising: an intranasal locking connector, a nasal dropper, a syringe pump and a syringe. One end of the nasal dropper is movably set through the intranasal locking connector and nasal dropper is fixed by the intranasal locking connector, and the other end of the nasal dropper is connected to the output end of the syringe. The syringe is connected to the syringe pump.

Preferably, the intranasal locking connector includes a drip fitting, a locking joint and a hollow body, the drip fitting, the locking joint and the hollow body are provided with concentric through holes in the middle, the hollow body is provided in the locking joint, the hollow body is a material with elastic deformation, and the nasal dropper passes through the hollow body before locking and fits with or against the inner wall of the hollow body through holes, the drip fitting and the locking joint are detachably connected.

Preferably, the connection of the locking joint to the drip fitting comprises a threaded connection or a press-fit connection.

Preferably, the drip fitting and the locking joint can switch between the pre-locked state and the post-locked state by threaded connection. Before locking, the hollow body rotates with the locking joint during the circumferential rotation of the locking joint when the nasal dropper fits the inner wall of the hollow body through the hole with clearance. The nasal dropper rotates relative to the hollow body, or the nasal dropper and the hollow body remain relatively stationary and jointly rotate relative to the locking joint so that the nasal dropper does not rotate with the locking joint in the circumferential direction.

Preferably, before locking, when the nasal dropper is tightened against the inner wall of the hollow body, the static friction between the nasal dropper and the hollow body is greater than that between the hollow body and the locking joint in the circumferential direction. When the locking joint rotates in the circumferential direction, the static friction between the nasal dropper and the hollow body overcomes that between the hollow body and the locking joint, so that the hollow body is held still by the nasal dropper and does not rotate in the circumferential direction with the locking joint.

The distance from the lower surface of the hollow body to the upper limit surface in the natural state is greater than that between the bottom surface of the groove of the locking joint and the lower surface of the drip fitting passage tube when the locking joint and the drip fitting are locked. This upper limit surface is the upper surface or shoulder of the hollow body.

After locking, the hollow body is squeezed by the bottom surface of the groove and the lower surface of the passage tube together and causes a tendency to expand in the radial direction, so that the nasal dropper, the hollow body and the locking joint are fixed. The static friction between the upper limit surface of the hollow body and the lower end surface of the passage tube reaches the most. The static friction between the lower end surface of the hollow body and the groove bottom surface of the locking joint is at its maximum.

Preferably, one end of the nasal dropper is sealed and a drip hole is provided on the side wall. The other end of the nasal dropper is provided with a nasal dropper connector. The nasal dropper connector is connected to the output end of the syringe. The outer wall of the nasal dropper is provided with a scale. The scale of the nasal dropper is provided at the end close to the drip fitting. A rigid wire is detachably provided inside the nasal dropper, which can be loaded and removed from the end of the nasal dropper near the syringe.

Preferably, wherein the locking joint is provided with a groove near the drip fitting, and the hollow body is provided in the groove. A passage tube is provided at the end of the locking joint near the drip fitting. When the drip fitting and the locking joint are connected, the passage tube is tightened against one end of the hollow body to achieve a coaxial relationship with the hollow body, and the aperture diameter in the middle of the passage tube is larger than that of the through hole of the hollow body.

Preferably, the nasal drug delivery device further comprises an intranasal connector, the intranasal connector is a four-way connector having a lower hole and three branches, wherein the lower hole is connected to a drip fitting and the intranasal connector extends into the nasal cavity for drug delivery instead of the drip connector. The three branches are an upper branch, a left branch and a right branch, wherein the upper branch is an intranasal branch, and the intranasal branch is provided with a guide spacer near the top end to guide the nasal dropper towards the nasal flank side.

Preferably, the nasal dropper can also be provided with a limiting structure that can be detachably connected to the nasal dropper by gluing, threading, or clamping. In addition, the diameter length of the limiting structure can be greater than the diameter length of the through hole formed by the drip fitting, the hollow body and the locking joint.

Compared with the prior art, the present disclosure has the following beneficial effects:

The present disclosure uses a split structure of a nasal dropper and an intranasal locking connector, The nasal dropper is movably set in the intranasal locking connector and fixed by the intranasal locking connector, and the intranasal locking connector is fixed under the user's nasal cavity, By adjusting the length of the nasal dropper into the nasal cavity, the drip position can be adjusted to suit different patients.

The present disclosure is simple in structure, convenient in use and reasonable in design. The hollow body placed in the intranasal locking connector can both fix the nasal dropper and make the medicine pass through the nasal dropper.

In the present disclosure, the scale on the nasal dropper disclosed is used to record the relative position of the nasal dropper to the intranasal locking connector. The drip hole of the nasal dropper can be accurately placed in the nasal olfactory area by simply connecting the locking connector at a specific position of the nasal dropper, thereby eliminating the need to place the nasal dropper with the aid of an endoscope each time it is put on.

The present disclosure has a removable rigid wire in the nasal dropper to facilitate the nasal entry operation. The rigid wire is removed after the nasal drip operation is completed, so that the nasal dropper can be restored to flexibility.

### BRIEF DESCRIPTION OF THE DRAWINGS

Other features, objects and advantages of the present disclosure will become more apparent from a reading of the detailed description of non-limiting embodiments made with reference to the following accompanying drawings:

FIG. 1 is a schematic diagram of the overall structure of the nasal drug delivery device in this disclosure after being worn by the user;

FIG. 2 a schematic diagram of the nasal drug delivery device disclosed in Example 1 of this disclosure;

FIG. 3 is a schematic diagram of the nasal drug delivery device disclosed in Example 2 of this disclosure;

FIG. 4 a cross-sectional view of the intranasal connector in this disclosure;

FIG. 5 is a schematic diagram of the overall structure of the wearable device in this disclosure for highlighting.

### ATTACHED MARK DESCRIPTION

| | |
|---|---|
| intranasal connector 110 | Bottom Surface of the Groove 126 |
| Guide Spacer 111 | Wearable Device 130 |
| intranasal locking connector 120 | Tube Hose 131 |
| drip Fitting 121 | Locking Clasp 132 |
| locking joint 122 | Two-way Connector 133 |
| Hollow Body 123 | Nasal Dropper 200 |
| Passage Tube 124 | Syringe Pump 300 |
| Lower Surface of Passage Tube 125 | Syringe 400 |

### DETAILED DESCRIPTION

The present disclosure is described in detail below in connection with specific embodiments. The following embodiments will assist those technicians in the art in further understanding the present disclosure, but do not limit the disclosure in any form. It should be noted that to ordinary technicians in the art, a number of variations and improvements can be made without departing from the conception of the present disclosure. These all belong to the scope of protection of the present disclosure.

### Embodiment 1

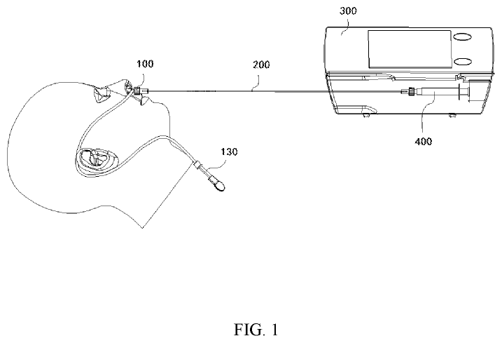

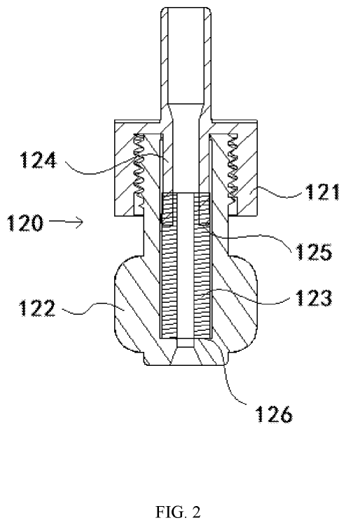

The present disclosure discloses a nasal drug delivery device with adjustable position, referring to FIG. 1 and FIG. 2, comprising: an intranasal connector 120, a nasal dropper 200, a syringe pump 300, and a syringe 400. One end of the nasal dropper 200 is movably set through the intranasal locking connector 120 and the nasal dropper 200 is secured by the intranasal locking connector 120, other end of the nasal dropper 200 is connected to the output end of the syringe400. The syringe 400 is connected to the syringe pump 300.

The intranasal locking connector 120 includes a drip fitting 121, a locking joint 122 and a hollow body 123; the drip fitting 121 is used to extend into the patient's nasal cavity for drug administration, and the intranasal locking connector 120 is secured to the lower edge of the patient's nasal cavity by attaching a wearable device to the intranasal locking connector 120. The drip fitting121, the locking joint 122 and the hollow body 123 are provided with concentric holes in the middle, and the hollow body 123 is rotatable in the locking joint 122, and the hollow body 123 is a material with elastic deformation, which can be made of silicone, rubber and foam, etc. The drip fitting 121 and the locking joint 122 are removably connected, for example, the connection between the drip fitting 121 and the locking joint 122 is a threaded connection; in another specific embodiment of the disclosure, the connection between the drip fitting 121 and the locking joint 122 is a press-fit connection. The nasal dropper 200 is placed in the nasal cavity through the through hole of the intranasal locking connector 120.

Since the nasal dropper 200 is soft and has a smooth wall, it is not easy to be clamped, and the flow of liquid in the nasal dropper 200 will be affected when the clamping force is too strong. In this disclosure, the drip fitting 121 and the locking joint 122 are used to cooperate, and the nasal dropper 200 passes through the flexible hollow body 123 and is restrained, so that the nasal dropper 200 entering the nasal cavity is kept fixed, solving the technical problem of the prior art that the hose is difficult to be fixed technical difficulties of the prior art, realizing the ability to keep the medicine through the nasal dropper 200 while keeping the incoming nasal tube fixed in the nasal olfactory area for a long time. Labor costs are saved, and most importantly, the rate of drug deposition in the nasal olfactory area is improved.

The locking joint 122 is provided with a recess at one end near the drip fitting 121, the hollow body 123 is provided in the recess, and a passage tube 124 is provided at one end of the drip fitting 121 near the locking joint 122, and after the drip fitting 121 and the locking joint 122 are connected, the passage tube 124 is tightened against one end of the hollow body, and the passage tube 124 is coaxial with the hollow body 123, and the aperture diameter of the middle of the passage tube 124 is larger than the aperture diameter of the hollow body 123. When installing or replacing the hollow body 123, it is only necessary to place the new hollow body 123 in the recess of the locking joint 122 and then screw the locking joint 122 back onto the drip fitting 121. After tightening, the passage tube 124 on the drip fitting 121 is tightened against the hollow body 123, thus fixing the hollow body 123, and the nasal dropper 200 enters through the through hole of the locking joint 122, passes through the through hole of the hollow body 123 and the through hole of the passage tube 124, and then passes out from the other end of the drip fitting 121. More importantly, when the passage tube 124 is tightened against the hollow body 123, the hollow body has a certain elasticity, which makes the connection between the drip fitting 121 and the locking joint 122 tighter and has a certain anti-loosening effect.

The drip fitting 121 and the locking joint 122 can switch between the pre-locking state and the post-locking state, before locking, the nasal dropper 200 passes through the hollow body 123 and fits with the inner wall of the through hole of the hollow body 123 with a gap or against the tightness, when the nasal dropper 200 fits with the hollow body 123 with a gap, the hollow body 123 rotates with the locking joint 122 when the locking joint 122 is rotated circumferentially, and the nasal dropper 200 and the hollow body 123 are rotated, or the nasal dropper 200 and the hollow body 123 remain relatively stationary and rotate together with the locking joint 122, so that the nasal dropper 200 does not rotate circumferentially with the locking joint 122.

When the nasal dropper 200 is tightened against the inner wall of the hollow body 123, the static friction between the nasal dropper 200 and the hollow body 123 is greater than the static friction between the hollow body 123 and the locking joint 122 in the circumferential direction; when the locking joint 122 rotates circumferentially, the static friction between the nasal dropper 200 and the hollow body 123 overcomes the static friction between the hollow body 123 and the locking joint 122. so that the hollow body 123 is held still by the nasal dropper 200 and does not rotate circumferentially with the locking joint 122.

The distance from the lower end surface of said hollow body 123 to the upper limit surface in the natural state is greater than the distance between the bottom surface of the groove 126 of the locking joint 122 and the lower surface of passage tube 125 when the locking joint 122 is locked with the drip fitting 121, and the upper limit surface is the upper end surface or shoulder of the hollow body 123.

After locking, the hollow body 123 is subjected to a tendency to expand in the radial direction caused by the bottom surface of the groove 126 and the lower surface of passage tube 125, causing the three the nasal dropper 200, the hollow body 123 and the locking joint 122 to fix, and the static friction between the upper end limiting surface of the hollow body 123 and the lower surface of passage tube 125 is maximized, The static friction between the lower surface of the hollow body 123 and the bottom surface of the groove 126 of the locking joint 122 is maximized.

One end of the nasal dropper 200 is sealed, and the side wall is provided with a drip hole for dripping medicine into the nasal olfactory area, and the shape of the drip hole is straight round with a diameter of 0.4˜0.6 mm, and the position of the center of the circle of the drip hole is 1 mm from the end, so that when the nasal dropper is inserted into the nasal cavity, the direction of the open hole can be visually identified, and the orientation of the open hole can be positioned before re-insertion according to the operation requirements during use, so that the drip hole is accurately The other end of the nasal dropper 200 can be visually identified. The other end of the nasal dropper 200 is provided with a nasal dropper connector, and the nasal dropper connector is connected to the output end of the syringe 400.

The nasal dropper 200 is provided with a scale on the outer wall, the scale of the nasal dropper 200 is provided at the end near the drip fitting 121, and the scale index of the nasal dropper 200 is 2 mm.

The nasal dropper 200 is detachably provided with rigid wire inside to facilitate nasal entry operation. The rigid wire is loaded and removed from the end of the nasal dropper 200 near the syringe 400, and the rigid wire in this disclosure can be a stainless steel wire, or a fine wire made of rigid plastic, or other fine wire, to the extent that it is easy to enter the nose operation.

### Embodiment 2

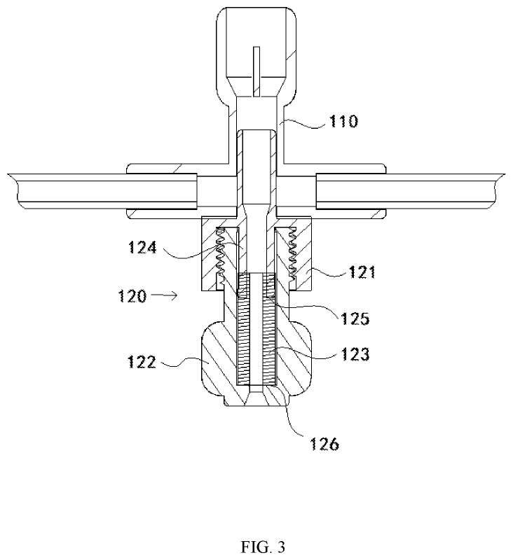

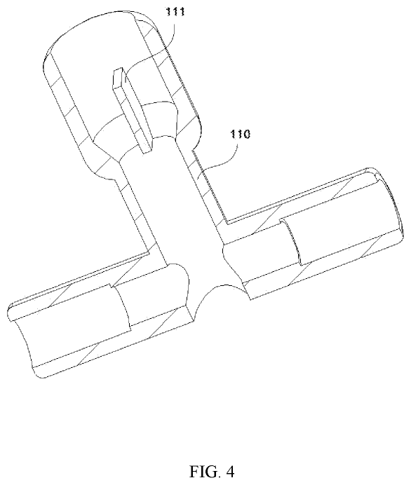

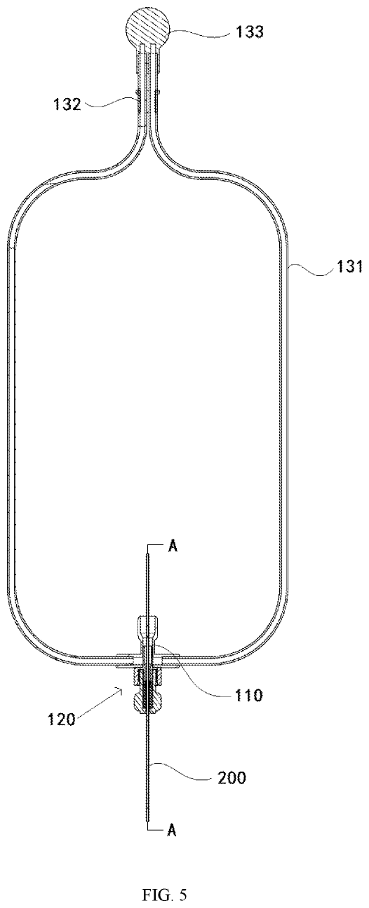

This embodiment is a preferred example of Example 1, specifically, in this embodiment, the nasal drug delivery device further comprises an intranasal connector 110, with reference to FIGS. 3 and 4, the intranasal connector 110 is a four-way connector having a lower hole and three branches, wherein the lower hole is connected to a drip fitting 121 and the intranasal connector extends into the nasal cavity for drug delivery instead of the drip connector. The three branches are an upper branch, a left branch and a right branch, wherein the upper branch is an intranasal branch, and the intranasal branch is provided with a guide spacer 111 near the top end to guide the nasal dropper 200 towards the nasal flank side. Referring to FIG. 5, said left and right branches are connected to a wearable device 130, the wearable device 130 being used to secure the intranasal connector 110 to the lower edge of the nose. In this embodiment, the intranasal connector 110 component is transparent light green and can be visually observed to adjust the insertion direction of the nasal dropper 200 biased to the intended side.

### Embodiment 3

In a preferred embodiment, the nasal dropper 200 can also be provided with a limiting structure that can be detachably connected to the nasal dropper 200 by gluing, threading or clamping, and the purpose of the limiting structure is to have a positioning effect, preferably, when the patient is administered for the second time, the medical staff can confirm the depth of nasal dropper 200 according to the scale recorded on the surface of nasal dropper 200 when the same patient enters the nasal olfactory area for the first time. Due to the relatively dense scale, it is cumbersome to take into account the depth of nasal dropper 200 and the opening direction of the nasal dropper 200. Therefore, the limiting structure is set at the scale of the nasal dropper 200 recorded for the first time in the same patient, and has the function of indicating the scale. In addition, the diameter length of the limiting structure can be greater than the diameter length of the through hole formed by the drip fitting 121, the hollow body 123 and the locking joint 122, so as to play the limiting role; In addition, the limiting structure has an indicative role, and the limiting structure can be set to a reasonable shape to indicate the opening direction of the nasal dropper 200, reducing the complexity of the operation of medical staff. In this disclosure, the limiting structure may be arranged in any shape for the purpose of indicating the scale, limiting role and indicating the open direction of the nasal dropper 200, where the specific shape of the limiting structure is not limited.

Referring to FIGS. 4 and 5, in one embodiment of the present disclosure, the wearable device 130 includes a tube hose 131, a locking clasp 132 and a two-way connector 133, two hoses 131 are connected to the left branch and the right branch of the inlet connector 110, and the other ends of the two hoses 131 are connected to the two-way connector 133 after passing through the locking clasp 132, and the hose 131 and the two-way connector 133 can be connected by the hose 131 and the two-way connector 133 can be bonded together. The locking clasp 132 may be a ring, or other structure capable of locking, to the extent that it is capable of securing the wearable device 130.

In an embodiment of the present disclosure, the wearable device 130 comprises a fixing band and a velcro, the fixing band being connected to the left and right branch tubes of the inlet connector, said velcro being located at the other end of the fixing band.

In one embodiment of the disclosure, the wearable device 130 is an elastic ring-shaped fixation band, and the two ends of said ring-shaped fixation band are connected to the left and right branch tubes of the intranasal connector 110, respectively.

This disclosure also discloses a method of using a drug delivery position adjustable nasal drug delivery device, based on the aforementioned drug delivery position adjustable nasal drug delivery device, comprising the steps of.

Step S1: Remove the nasal dropper 200, insert the graduated end of the nasal dropper 200 through the intranasal locking connector 120 and the intranasal connector 110, while keeping the opening of the nasal dropper 200 oriented toward the nasal flank side, and slowly insert it into the user's nasal cavity along the user's nasal area;

Step S2: after checking the insertion of the nasal dropper 200 into place using the endoscope, grasp the nasal dropper 200 by hand to keep it still and slide the intranasal connector 110 in the direction of the user's nasal cavity until the user's nasal cavity is reached;

Step S3: locking the intranasal locking connector 120, during the locking, the nasal dropper 200 and the locking joint 122 can be rotated relative to each other, after the locking, the hollow body 123 is tightened by the groove of the locking joint 122 and the passage tube 124 of the drip fitting 121, so that the nasal dropper 200 is fixed in the intranasal locking connector 120, at this time the nasal dropper 200 has been fixed in the nasal cavity, using the wearable device 130 to fix the intranasal connector 110 to the lower edge of the user's nasal cavity, recording the scale of the nasal dropper 200;

Before locking, the nasal dropper 200 passes through the hollow body 123 and then fits the inner wall of the hollow body 123 with gap or against the inner wall of the hollow body 123. When the nasal dropper 200 fits the hollow body 123 with gap, the hollow body 123 rotates with the locking joint 122 when the locking joint 122 rotates circumferentially, and the nasal dropper 200 rotates relatively with the hollow body 123, or the nasal dropper 200 and the hollow body 123 remain relatively stationary and rotate relatively with the locking joint 122 together, so that the nasal dropper 200 does not rotate circumferentially with the locking joint 122.

When the nasal dropper 200 is pressed against the inner wall of the hollow body 123, the static friction between the nasal dropper 200 and the hollow body 123 is greater than the static friction between the hollow body 123 and the locking joint 122 in the circumferential direction; when the locking joint 122 rotates circumferentially, the static friction between the nasal dropper 200 and the hollow body 123 overcomes the static friction between the hollow body 123 and the locking joint 122 so that the hollow body 123 is held still by the nasal dropper 200 and does not rotate circumferentially with the locking joint 122.

The distance from the lower face of said hollow body 123 to the upper limiting face in its natural state is greater than the distance between the bottom surface of the groove 126 of the locking joint 122 and the lower surface of the passage tube 125 of the drip fitting 121 when the locking joint 122 is locked with the drip fitting 121, said upper limiting face being the upper face or shoulder of the hollow body 123.

After locking, the hollow body 123 is subjected to a tendency to expand in the radial direction caused by the bottom surface of the groove 126 and the lower surface of passage tube 125, causing the three the nasal dropper 200, the hollow body 123 and the locking joint 122 to fix, and the static friction between the upper end limiting surface of the hollow body 123 and the lower surface of passage tube 125 is maximized, The static friction between the lower surface of the hollow body 123 and the bottom surface of the groove 126 of the locking joint 122 is maximized.

Step S4: Remove the rigid wire in the nasal dropper 200, insert the other end of the nasal dropper 200 into the connector of the nasal dropper 200 and lock it, and screw the connector of the nasal dropper 200 to the syringe 400.

Step S5: Operate the syringe pump 300, set the fixed volume of injection, said fixed volume is the fixed volume capacity of the nasal dropper 200, and fill the nasal dropper 200 with the medicine solution.

Step S6: The syringe pump 300 is set up to administer the drug, and the syringe pump 300 is used to administer the drug until the drug drip is completed, and the syringe pump 300 beeps after the drug drip is completed.

Wherein, recording the scale on the nasal dropper 200 in step S3 allows the next insertion of the nasal dropper 200 without the use of an endoscope.

The syringe 400 and syringe pump 300 may be commercially available devices to the extent that they are adequate for drug administration.

In the description of this application, it should be understood that the terms “up”, “down”, “front”, “back”, “left”, “right”, “vertical”, “horizontal”, “top”, “bottom”, “inside”, “outside”, etc. indicate a bearing or position relationship based on the bearing or position relationship shown in the attached drawings and are intended only to facilitate the description of this application and simplify the description. Rather than indicating or implying that the device or element referred to must have a particular orientation, be constructed and operated in a particular orientation and therefore cannot be construed as a limitation of this application.

Specific embodiments of the present disclosure have been described above. It is to be understood that the present disclosure is not limited to the above embodiments, and that various variations or modifications can be made by those skilled in the art within the scope of the claims, which do not affect the substance of the present disclosure. Without conflict, the embodiments, and features of the embodiments of the present application may be arbitrarily combined with each other.

1. A nasal drug delivery device with adjustable position, characterized in that the nasal drug delivery device comprises: an intranasal locking connector, a nasal dropper, a syringe pump and a syringe, wherein one end of the nasal dropper is movably set through the intranasal locking connector and the nasal dropper is fixed by the intranasal locking connector, and the other end of the nasal dropper is connected to the output end of the syringe, wherein the syringe is connected to the syringe pump.

2. The nasal drug delivery device with adjustable position according to claim 1, characterized in that: wherein the intranasal locking connector comprises a drip fitting, a locking joint, and a hollow body, wherein the drip fitting, the locking joint and the hollow body are provided with concentric through-holes in the middle of each other, wherein the hollow body is provided in the drip fitting, the hollow body has a material with elastic deformation, and the nasal dropper passes through the hollow body before locking and fits with or against an inner wall of the hollow body through holes, the drip fitting and the locking joint are detachably connected.

3. The nasal drug delivery device with adjustable position according to claim 2, characterized in that the connection of the locking joint to the drip fitting comprises a threaded connection or a press-fit connection.

4. The nasal drug delivery device with adjustable position according to claim 3, characterized in that the drip fitting and the locking joint switch between a pre-locked state and a post-locked state by the threaded connection, wherein before locking, the hollow body rotates with the locking joint during a circumferential rotation of the locking joint when the nasal dropper fits the inner wall of the hollow body through the holes with clearance, wherein the nasal dropper rotates relative to the hollow body, or the nasal dropper and the hollow body remain relatively stationary and jointly rotate relative to the locking joint so that the nasal dropper does not rotate with the locking joint in a circumferential direction.

5. The nasal drug delivery device with adjustable position according to claim 4, characterized in that, before locking, when the nasal dropper is tightened against the inner wall of the hollow body, a static friction between the nasal dropper and the hollow body is greater than that between the hollow body and the locking joint in the circumferential direction, when the locking joint rotates in the circumferential direction, the static friction between the nasal dropper and the hollow body overcomes that between the hollow body and the locking joint, so that the hollow body is held still by the nasal dropper and does not rotate in the circumferential direction with the locking joint,
wherein the distance from a lower surface of the hollow body to an upper limit surface in the natural state is greater than that between a bottom surface of the groove of the locking joint and a lower surface of the drip fitting passage tube when the locking joint and the drip fitting are locked, wherein the upper limit surface is the upper surface or shoulder of the hollow body,
wherein after locking, the hollow body is squeezed by the bottom surface of the groove and the lower surface of the passage tube together and causes a tendency to expand in a radial direction, so that the nasal dropper, the hollow body and the locking joint are fixed, wherein the static friction between the upper limit surface of the hollow body and the lower end surface of the passage tube reaches the most, wherein the static friction between the lower end surface of the hollow body and the groove bottom surface of the locking joint is at its maximum.

6. The nasal drug delivery device with adjustable position according to claim 1, characterized in that, one end of the nasal dropper is sealed and a drip hole is provided on the side wall, wherein the other end of the nasal dropper is provided with a nasal dropper connector, wherein the nasal dropper connector is connected to the output end of the syringe, the outer wall of the nasal dropper is provided with a scale, wherein the scale of the nasal dropper is provided at the end close to the drip fitting, a rigid wire is detachably provided inside the nasal dropper, which is loaded and removed from the end of the nasal dropper near the syringe.

7. The nasal drug delivery device with adjustable position according to claim 1, characterized in that, the locking joint is provided with a groove near the drip fitting, and the hollow body is provided in the groove, a passage tube is provided at the end of the locking joint near the drip fitting, wherein when the drip fitting and the locking joint are connected, the passage tube is tightened against one end of the hollow body to achieve a coaxial relationship with the hollow body, and an aperture diameter in the middle of the passage tube is larger than that of the through hole of the hollow body.

8. The nasal drug delivery device with adjustable position according to claim 1, characterized in that, wherein the nasal drug delivery device further comprises an intranasal connector, the intranasal connector is a four-way connector having a lower hole and three branches, wherein the lower hole is connected to a drip fitting and the intranasal connector extends into the nasal cavity for drug delivery instead of the drip connector, wherein the three branches are an upper branch, a left branch and a right branch, wherein the upper branch is an intranasal branch, and the intranasal branch is provided with a guide spacer near the top end to guide the nasal dropper towards a nasal flank side.

9. The nasal drug delivery device with adjustable position according to claim 1, characterized in that, wherein the nasal dropper is provided with a limiting structure that is detachably connected to the nasal dropper by gluing, threading or clamping.

10. The nasal drug delivery device with adjustable position according to claim 9, characterized in that, the diameter length of the limiting structure being greater than the diameter length of the through hole formed by the drip fitting, the hollow body and the locking joint.
